(19) United States
(12) Patent Application Publication
(10) Pub. No.: US 2022/0351383 A1
(43) Pub. Date: Nov. 3, 2022

(54) METHODS AND APPARATUS FOR DYNAMIC IMAGING

(71) Applicant: Spectral Instruments Imaging, LLC, Tucson, AZ (US)

(72) Inventor: Michael Bo Nelson, Tucson, AZ (US)

(21) Appl. No.: 17/621,443

(22) PCT Filed: Jun. 16, 2020
(86) PCT No.: PCT/US2020/037867

§ 371 Date: Dec. 21, 2021

Related U.S. Application Data
(60) Provisional application No. 62/866,382, filed on Jun. 25, 2019.

(51) Int. Cl. G06T 7/00; G06V 10/75; G06V 10/25

(52) U.S. Cl. CPC G06T 7/0016; G06V 10/759; G06V 10/25; A61B 5/0077

(57) ABSTRACT

A method is provided herein that discloses receiving, by a processor, first image data comprising first bioluminescence data derived from an organism at a first time, receiving, by the processor, second image data comprising second bioluminescence data derived from an organism at a second time, comparing, by the processor, the first image data to the second image data, determining, by the processor, whether a peak light output event has occurred based on the comparison, and outputting, by the processor and to a display device, an indication that the peak light output event has occurred.

### FIELD

The present disclosure relates to imaging systems, particularly Bioluminescence Imaging.

### BACKGROUND

Bioluminescence Imaging (BLI) is a widely used biological tracking method for the progression of disease or a pathogen within a live animal. This technique involves the pathogen or cell line (often cancer cells) to be attached with a gene to allow the target to emit light and a very sensitive optical imaging system to view the signal. During the process, a substrate called luciferin is injected into the animal before imaging. When the luciferin reaches the cell, and in conjunction with oxygen and adenosine triphosphate (ATP), light is created. This light output increases for a period of time, generally plateaus for a period of time, then begins to decrease as the luciferin enzyme is metabolized. The peak light output is typically valued from a research standpoint.

### SUMMARY

In various embodiments, a method is provided comprising receiving, by a processor, first image data comprising first bioluminescence data derived from an organism at a first time, receiving, by the processor, second image data comprising second bioluminescence data derived from an organism at a second time, comparing, by the processor, the first image data to the second image data, determining, by the processor, whether a peak light output event has occurred based on the comparison, and outputting, by the processor and to a display device, an indication that the peak light output event has occurred.

In various embodiments, an article of manufacture is provided including a non-transitory, tangible computer readable storage medium having instructions stored thereon that, in response to execution by a computer-based system, cause the computer-based system to perform operations comprising receiving, by the computer-based system, first image data comprising first bioluminescence data derived from an organism at a first time, receiving, by the computer-based system, second image data comprising second bioluminescence data derived from an organism at a second time, comparing, by the computer-based system, the first image data to the second image data, determining, by the computer-based system, whether a peak light output event has occurred based on the comparison, and outputting, by the computer-based system and to a display device, an indication that the peak light output event has occurred.

In various embodiments, a system is provided comprising, a processor, an optical imaging device, a display device, the processor in logical communication with the optical imaging device and the display device, and a tangible, non-transitory memory configured to communicate with the processor, the tangible, non-transitory memory having instructions stored thereon that, in response to execution by the processor, cause the processor to perform operations comprising, receiving, by the processor, first image data comprising first bioluminescence data derived from an organism at a first time, receiving, by the processor, second image data comprising second bioluminescence data derived from an organism at a second time, comparing, by the processor, the first image data to the second image data, determining, by the processor, whether a peak light output event has occurred based on the comparison, and outputting, by the processor and to the display device, an indication that the peak light output event has occurred.

The foregoing features, elements, steps, or methods may be combined in various combinations without exclusivity, unless expressly indicated herein otherwise. These features, elements, steps, or methods as well as the operation of the disclosed embodiments will become more apparent in light of the following description and accompanying drawings.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements.

FIG. 1 illustrates a schematic representation of an optical imaging system, in accordance with various embodiments.

FIG. 2 illustrates a further view of the optical imaging system of FIG. 1, in accordance with various embodiments.

FIG. 3 illustrates image data, in accordance with various embodiments.

FIG. 4 illustrates further image data, in accordance with various embodiments.

FIG. 5 illustrates a method of image analysis, in accordance with various embodiments.

FIG. 6 illustrates a method of image analysis, in accordance with various embodiments.

FIG. 7 illustrates a method of image analysis, in accordance with various embodiments.

FIG. 8 illustrates a method of image analysis, in accordance with various embodiments.

FIG. 9 illustrates light output intensity of various regions of interest over time, in accordance with various embodiments; and

FIG. 10 illustrates light output intensity curve over time at given time intervals, in accordance with various embodiments.

### DETAILED DESCRIPTION

The detailed description of exemplary embodiments herein makes reference to the accompanying drawings, which show exemplary embodiments by way of illustration. While these exemplary embodiments are described in sufficient detail to enable those skilled in the art to practice the inventions, it should be understood that other embodiments may be realized and that logical changes and adaptations in design and construction may be made in accordance with this invention and the teachings herein. Thus, the detailed description herein is presented for purposes of illustration only and not of limitation. The scope of the invention is defined by the appended claims. For example, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Surface shading and/or crosshatching lines may be used throughout the figures to denote different parts, but not necessarily to denote the same or different materials.

BLI uses an optical imaging system to capture image data. The image data often relates to a subject organism, such as a mouse. The image data contains a representation of the intensity of light output from the organism due to the metabolism of luciferin. The metabolism may be performed by tumor cells or other cells of the organism and the resultant light output may be sensed by an optical imaging system. The peak light output, which may be sustained for a short time (or, “plateau”), is useful for research purposes. Often, for ease of operation, image data is captured a set time after luciferin administration, and the light output intensity is deemed the peak light output. In many cases, this set time is 10 minutes.

In accordance with various aspects, a BLI system is provided for image capture. In various embodiments, light production measurements produced from light production from the subject organism, though light production can vary from a myriad of issues such as tumor size, total tumor burden, the time of administration, location of the organism's body, the health of the organism and temperature, among other factors.

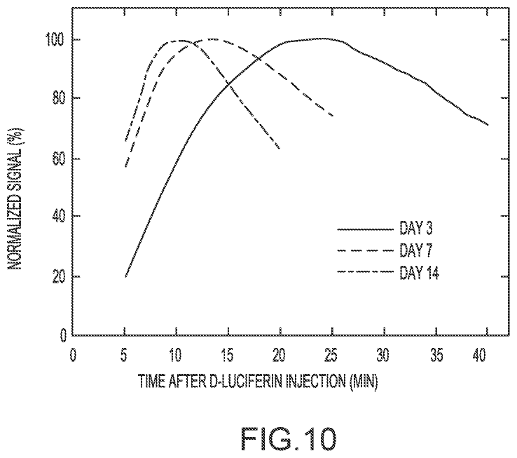

With reference to FIG. 10, for example, a light output intensity curve over time at given time intervals for the same organism. The normalized signal (i.e., light output intensity) is given in the y axis and time is given on the x axis. As shown, for Days 3 and 7, the peak light output intensity is observable at ten minutes. However, on Day 14, the peak light output intensity occurs closer to 20 minutes. A reading taken at ten minutes would yield an artificially low peak light output intensity, which negatively affects the quality of data derived from the study. However, if one were to take image data at short intervals for an extended period of time, one would likely cause the study to consume too much time due to continuing to take image data after the peak light output intensity occurs. This would lead to wasted experimental throughput, as well as extended anesthesia times for the organisms.

In that regard, in various aspects, image data is taken from an optical imaging system and compared to previous image data. In various embodiments, a peak light output intensity may be determined and compared between different sets of image data. Once a peak light output intensity is found, the measurement may be ended. In other embodiments, one or more regions of interest (ROI) may be defined. Each ROI may be compared separately across dets of image data, and thus peak light output intensity may be determined per ROI.

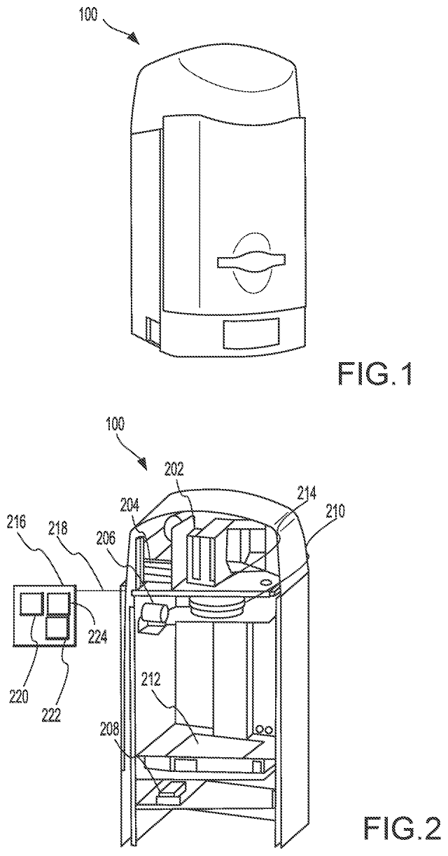

With reference to FIG.1, optical imaging system 100 is shown. Optical imaging system 100 may be any system capable of capturing image data. With reference to FIG. 2, optical imaging system 100 is shown without the light tight door. Camera 202 may be referred to as an optical imaging device. Camera 202 is mounted to view stage 212 through lens array 210 and filter 214. X-ray camera 206 may also be present, as well as translating x-ray source 208. Camera 202 may comprise any suitable optical sensor. For example, camera 202 may comprise any optical sensor that is capable of sensing visible light. In various embodiments, camera 202 comprises a charged-coupled device (CCD) and/or a complementary metal-oxide-semiconductor (CMOS). In various embodiments, camera 202 comprises a cooling system to cool an optical sensor to a predetermined temperature. This this cooling may reduce image background noise generally associated with higher temperatures. The cooling system may use air or liquid to cool the sensor. For example, camera 202 may comprise a CCD operable to be cooled to between −100° C. and −80° C. and have a size of 27.6×27.6 mm, though any suitable size is contemplated herein. Lens array 210 may comprise any suitable lens that is at least partially transmissive to visible light. Lens array 210 may have any suitable focal length, for example, that between 15 mm and 100 mm, though in various embodiments lens array has a focal length of 50 mm and a maximum aperture of f/1.2. Filter 214 may comprise any suitable filter arrangement. For example, filter 214 may comprise one or more excitation light emitting diodes (LEDs) of wavelengths of 360, 405, 430, 465, 500, 535, 570, 605, 640, 675, 710, 745, and 770 nm, or any other suitable wavelength. Filter 214 may comprise one or more emission filters of wavelengths 490, 510, 530, 550, 570, 590, 610, 630, 650, 670, 690, 710, 730, 750, 770, 790, 810, 830, 850, and 870 nm, or any other suitable wavelength. Electronic control device 204 may comprise one or more processors, computer-based systems, tangible memories, computer readable storage media, and/or other electronics to operate optical imaging system 100. A processor(s) and/or tangible memories may be housed within optical imaging system 100 in electronic control device 204 and configured to control optical imaging system 100. However, in various embodiments, such processor(s) and/or tangible memories may be housed separate from optical imaging system 100 and are connected to optical imaging system 100 via a logical connection. For example, computer-based system 216 is shown connecting to electronic control device 204 via logical interface 218. Computer based system 216 comprises one or more processors 220 and tangible memory 222. In that regard, optical imaging system 100 may be connected to a computer-based system 216 and via any method described herein, including, for example, through a serial bus, Ethernet connection, radio frequency connection, or other connection that allows the processor to communicate with optical imaging system 100.

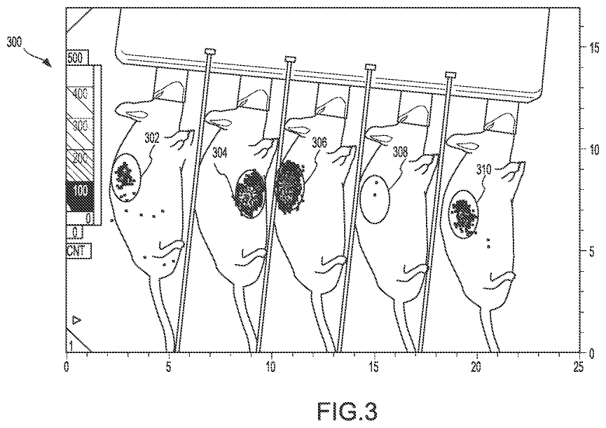

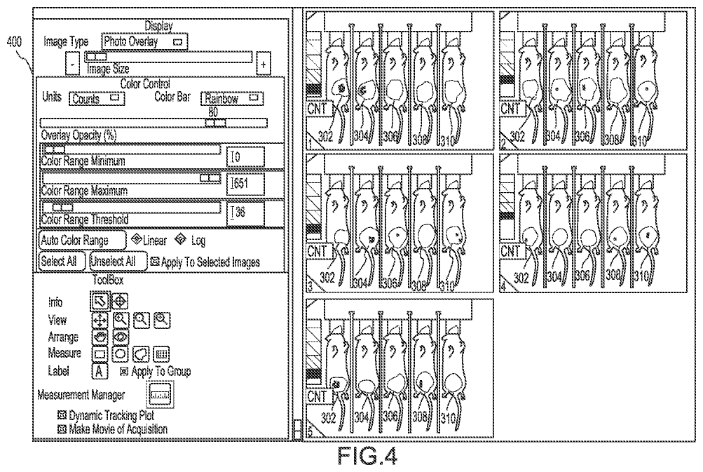

Image data 300 is shown with reference to FIG. 3. Image data 300 is shown in pictorial (i.e., rendered image) format, but it is understood that image data 300 may be stored in various data formats, including a RAW output of an optical sensor or a compressed format, whether lossless or “lossy.” Image data 300 may be taken by, for example, optical imaging system 100.

Image data 300 depicts five (5) anesthetized mice after administration of luciferin, though in various embodiments, image data 300 may represent images obtained after administration of any material that may cause bioluminescence. Regions of interest 302, 304, 306, 308 and 310 are defined relative to each mouse. Within each region of interest 302, 304, 306, 308 and 310, light intensity output is shown. The light intensity output is based upon the light intensity detected by optical imaging system 100.

Image data set 400 depicts multiple image data regarding five (5) anesthetized mice after administration of luciferin. Regions of interest 302, 304, 306, 308 and 310 are defined relative to each mouse. Regions of interest 302, 304, 306, 308 and 310 are shown in each image data 1, image data 2, image data 3, image data 4 and image data 5. Within each region of interest 302, 304, 306, 308 and 310, light intensity output is shown. The light intensity output is based upon the light intensity detected by optical imaging system 100.

In this regard, image data set 400 contains multiple image data taken at different time intervals as measured from the administration of luciferin. For example, the image data in image data set 400 may be taken from between 1 ms apart to 10 minutes apart, though in various embodiments, the image data is taken from between 15 seconds to 5 minutes apart, and in various embodiments, the image data is taken from between 30 seconds to 1 minute apart. In various embodiments, image data is taken continually. In other words, as soon as an image data is captured by optical imaging system 100, in embodiments where image data is taken continually, optical imaging system 100 begins to capture new image data.

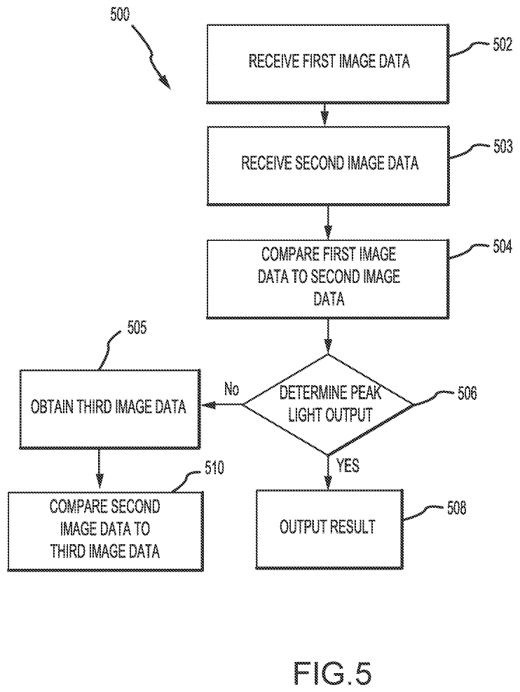

With reference to FIG. 5, method of image analysis 500 is illustrated. As discussed above, computer-based system 216 may comprise one or more processors 220 configured to receive image data. The processor 220 receives first image data (step 502), the first image data representing a bioluminescent organism. The processor receives second image data (step 503), the second image data representing a bioluminescent organism, the second image data taken at a time after first image data. The processor compares the first image data to the second image data (step 504). In step 504, the processor assesses first image data for a first light output intensity. The first light output intensity is representative of the light output from the organism. In various embodiments, the first light output intensity is determined using the average of a sample of the light emitting areas of the organism. In further embodiments, the first light output intensity is determined by adding the light intensity value of all light emitting points on the organism. In that regard, an organism with a low level of light output intensity on a large surface area may have the same light intensity output of an organism with a smaller surface area of light emitting points but having a higher light output intensity per point. The first light output intensity may further comprise average (arithmetic mean) of these values. Once obtained, the processor assesses second image data for a second light output intensity in a similar manner.

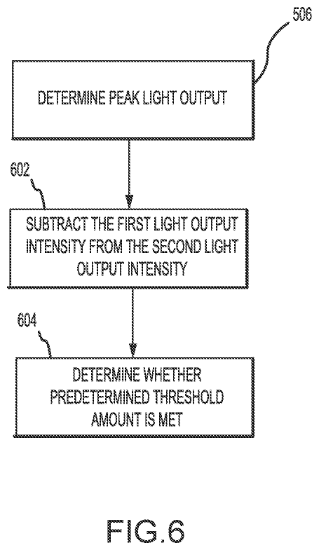

The processor may determine the peak light output (step 506), with additional reference to FIG. 6. In various embodiments, the processor may subtract the first light output intensity from the second light output intensity (step 602). The difference may then be used to determine the percentage change in light output intensity from first image data to second image data (step 604). If the difference is within a predetermined threshold amount, the processor may then determine that peak light output has been reached. For example, the predetermined threshold amount may be between 1% and 15%, between 2% and 10%, and between 5% and 8%. In this regard, if the light output intensity in second image data is within the predetermined threshold amount of the first image data, the processor may determine that peak light output has increased. Conversely, if the light output intensity in second image data exceeds the predetermined threshold amount of the first image data, the processor may determine that peak light output has not yet occurred.

With reference back to FIG. 5, if the peak output peak light output has not yet occurred, the processor may obtain third image data (step 505) and compare the second image data to third image data (step 510) in a manner similar to step 504. The obtaining of the third image data (step 505) may comprise commanding, by the processor, the optical imaging device to capture third image data. Image analysis may be repeated on an iterative basis, capturing many additional sets of image data. In various embodiments, even if the light output intensity in second image data is within the predetermined threshold amount of the first image data, additional imaging may be taken and analyzed prior to satisfying a confidence level that the peak light output has occurred. In various embodiments, when the light output intensity in second image data is within the predetermined threshold amount of the first image data, additional sets of image may be taken, for example, between zero and ten additional sets of image data. These additional sets of image data may be compared to the first image data, the second image data, or the additional sets of image data. In various embodiments, the number of additional sets of image data may be a variable parameter. However, in various embodiments, when the light output intensity in second image data is within the predetermined threshold amount of the first image data, zero additional image data sets may be taken.

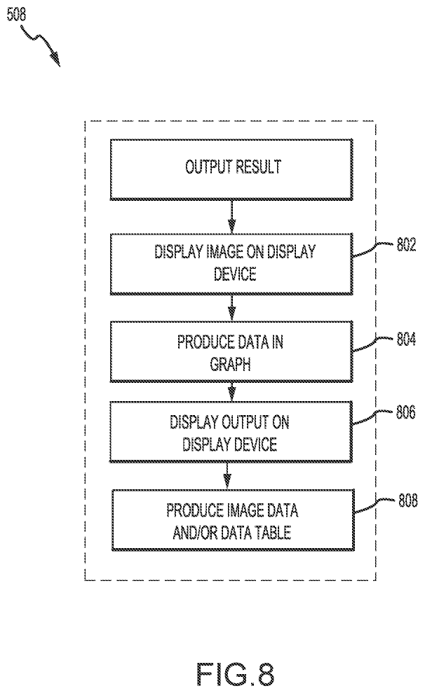

In response to peak light output being determined, and with reference to FIG. 8, the processor may output a result (step 508). Outputting the result may comprise, for example, graphing the light output over time (step 804), displaying the image data on a display device (step 802), indicating on a display device (for example, display device 224 with brief reference to FIG. 2) (step 806) that the peak light output has occurred, or any other suitable output. Outputting the result may also comprise, for example, producing (either on a display device or electronic file) image data (e.g., an image of the subject organism) and/or a data table (step 808). The data table contains, in various embodiments, light output intensity values, peak light output intensity values and the time the peak light output intensity values were measured.

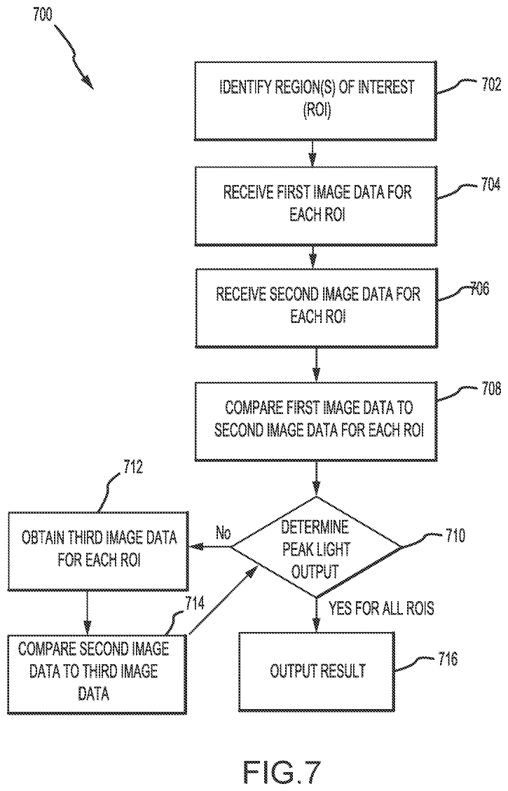

With reference to FIG. 7, method of image analysis 700 is illustrated. Method of image analysis 700 is similar to method of image analysis 500, but includes analyses per region of interest. As discussed above, computer-based system 216 may comprise one or more processors 220 configured to identify regions of interest (ROI) (step 702). The identification of an ROI may be informed by various factors. For example, the identification of ROI may be based on, for example, the organism being studied, the disease model, the particular study protocols, and other factors. For example, in a disease model examining cancer, the ROI may be defined to include portions of the cells exhibiting cancer. In various aspects, the ROI is selected using predetermined values relative to a field of view. Though, in further aspects, the ROI is based on predetermined values influenced by optical recognition of the organism's body by computer-based system 216. In that regard, computer-based system 216 may optically recognize the organism's body and identify the ROI using the predetermined values as applied to the organism's body. In various aspects, the ROI may be selected (whether arbitrarily or otherwise) through computer-based system 216 using a user interface such as a graphical user interface. In that regard, each ROI defines an area in the image data that is analyzed in subsequent process. The processor 220 receives first image data (step 704) for each ROI, the first image data representing a bioluminescent organism. The processor receives second image data (step 706), the second image data representing a bioluminescent organism, the second image data taken at a time after first image data. The processor compares the first image data to the second image data (step 708), comparing each ROI in the first image data to the same ROI in the second image data. In step 708, the processor assesses first image data for a first light output intensity. The first light output intensity is representative of the light output from the organism. In various embodiments, the first light output intensity is determined using the average of a sample of the light emitting areas of the organism within each ROI. In further embodiments, the first light output intensity is determined by adding the light intensity value of all light emitting points on the organism within each ROI. In that regard, an ROI on an organism with a low level of light output intensity on a large surface area may have the same light intensity output as an ROI of an organism with a smaller surface area of light emitting points but having a higher light output intensity per point. The first light output intensity may further comprise average (arithmetic mean) of these values. Once obtained, the processor assesses second image data for a second light output intensity in a similar manner.

The processor may determine the peak light output (step 710). In various embodiments, the processor may subtract the first light output intensity from the second light output intensity for each ROI. The difference may then be used to determine the percentage change in light output intensity from first image data to second image data for each ROI. If the difference is within a predetermined threshold amount, the processor may then determine that peak light output has been reached. For example, the predetermined threshold amount may be between 1% and 15%, between 2% and 10%, and between 5% and 8%. In this regard, if the light output intensity in second image data is within the predetermined threshold amount of the first image data, the processor may determine that peak light output has increased in the ROI of interest. Conversely, if the light output intensity in second image data exceeds the predetermined threshold amount of the first image data, the processor may determine that peak light output has not yet occurred in the ROI of interest.

With reference back to FIG. 7, if the peak output peak light output has not yet occurred in an ROI, the processor may obtain third image data (step 712) and compare the second image data to third image data (step 714) in a manner similar to step 708. The obtaining of the third image data (step 712) may comprise commanding, by the processor, the optical imaging device to capture third image data. The output of the comparison in step 714 is sent to step 710. In response to peak light output being determined, the processor may cease comparison in the ROI that has reached the peak light output but continue the process for ROIs that have not yet reached a peak light output. In that regard, steps 710, 712, and 714 function as a loop, which may continue for additional iterations until step 710 determines that the peak light output has occurred for all ROIs. Step 716 is reached when all ROIs have reached the peak light output.

In response to peak light output being determined for all ROIs, the processor may output a result (step 716). Outputting the result may comprise, for example, graphing the light output over time, displaying the image data on a display device, indicating on a display device (for example, display device 224 with brief reference to FIG. 2) that the peak light output has occurred, or any other suitable output.

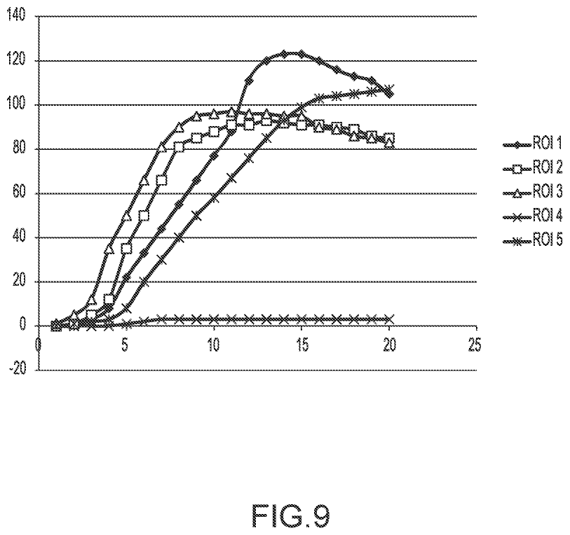

With brief reference to FIG.9, a graph of a sample output of process 700 is shown for ROI 1, ROI2, ROI 3, ROI4 and ROI 5. As shown, ROI 2 and ROI 3 peak near ten minutes from administration of the bioluminescent material. However, ROI 1 and ROI 4 peak closer to 13 or 14 minutes. In that regard, the peak values are more accurately determined, with little to no excess imaging time.

Computer programs (also referred to as computer control logic) are stored in main memory and/or secondary memory. Computer programs may also be received via communications interface. Such computer programs, when executed, enable the computer system to perform the features as discussed herein. In particular, the computer programs, when executed, enable the processor to perform the features of various embodiments. Accordingly, such computer programs represent controllers of the computer-based system.

These computer program instructions may be loaded onto a general-purpose computer, special purpose computer, computer-based system 216 or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer-based system or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

In various embodiments, software may be stored in a computer program product and loaded into a computer system using removable storage drive, hard disk drive, or communications interface. The control logic (software), when executed by the processor, causes the processor (for example, a processor 220) to perform the functions of various embodiments as described herein. In various embodiments, hardware components may take the form of application specific integrated circuits (ASICs). Implementation of the hardware state machine so as to perform the functions described herein will be apparent to persons skilled in the relevant art(s).

As will be appreciated by one of ordinary skill in the art, the system may be embodied as a customization of an existing system, an add-on product, a processing apparatus executing upgraded software, a stand-alone system, a distributed system, a method, a data processing system, a device for data processing, and/or a computer program product. Accordingly, any portion of the system or a module may take the form of a processing apparatus executing code, an internet-based embodiment, an entirely hardware embodiment, or an embodiment combining aspects of the internet, software, and hardware. Furthermore, the system may take the form of a computer program product on a computer-readable storage medium having computer-readable program code means embodied in the storage medium. Any suitable computer-readable storage medium may be utilized, including hard disks, CD-ROM, BLU-RAY DISC®, optical storage devices, magnetic storage devices, and/or the like.

In various embodiments, components, modules, and/or engines of optical imaging system 100 may be implemented as micro-applications or micro-apps. Micro-apps are typically deployed in the context of a mobile operating system, including for example, a WINDOWS® mobile operating system, an ANDROID® operating system, an APPLE® iOS operating system, a BLACKBERRY® company's operating system, and the like. The micro-app may be configured to leverage the resources of the larger operating system and associated hardware via a set of predetermined rules which govern the operations of various operating systems and hardware resources. For example, where a micro-app desires to communicate with a device or network other than the mobile device or mobile operating system, the micro-app may leverage the communication protocol of the operating system and associated device hardware under the predetermined rules of the mobile operating system. Moreover, where the micro-app desires an input from a user, the micro-app may be configured to request a response from the operating system which monitors various hardware components and then communicates a detected input from the hardware to the micro-app.

The system and method may be described herein in terms of functional block components, screen shots, optional selections, and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, C#, JAVA®, JAVASCRIPT®, JAVASCRIPT® Object Notation (JSON), VBScript, Macromedia COLD FUSION, COBOL, MICROSOFT® company's Active Server Pages, assembly, PERL®, PHP, awk, PYTHON®, Visual Basic, SQL Stored Procedures, PL/SQL, any UNIX® shell script, and extensible markup language (XML) with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JAVASCRIPT®, VBScript, or the like.

Accordingly, functional blocks of the block diagrams and flowchart illustrations support combinations of means for performing the specified functions, combinations of steps for performing the specified functions, and program instruction means for performing the specified functions. It will also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions. Further, illustrations of the process flows and the descriptions thereof may make reference to user WINDOWS® applications, webpages, websites, web forms, prompts, etc. Practitioners will appreciate that the illustrated steps described herein may comprise in any number of configurations including the use of WINDOWS® applications, webpages, web forms, popup WINDOWS® applications, prompts, and the like. It should be further appreciated that the multiple steps as illustrated and described may be combined into single webpages and/or WINDOWS® applications but have been expanded for the sake of simplicity. In other cases, steps illustrated and described as single process steps may be separated into multiple webpages and/or WINDOWS® applications but have been combined for simplicity.

For the sake of brevity, conventional data networking, application development, and other functional aspects of the systems (and components of the individual operating components of the systems) may not be described in detail herein. Furthermore, the connecting lines shown in the various figures contained herein are intended to represent exemplary functional relationships and/or physical couplings between the various elements. It should be noted that many alternative or additional functional relationships or physical connections may be present in a practical system.

In various embodiments, the methods described herein are implemented using the various particular machines described herein. The methods described herein may be implemented using the below particular machines, and those hereinafter developed, in any suitable combination, as would be appreciated immediately by one skilled in the art. Further, as is unambiguous from this disclosure, the methods described herein may result in various transformations of certain articles.

The various system components discussed herein may include one or more of the following: a host server or other computing systems including a processor for processing digital data; a memory coupled to the processor for storing digital data; an input digitizer coupled to the processor for inputting digital data; an application program stored in the memory and accessible by the processor for directing processing of digital data by the processor; a display device coupled to the processor and memory for displaying information derived from digital data processed by the processor; and a plurality of databases.

The present system or any part(s) or function(s) thereof may be implemented using hardware, software, or a combination thereof and may be implemented in one or more computer systems or other processing systems. However, the manipulations performed by embodiments were often referred to in terms, such as matching or selecting, which are commonly associated with mental operations performed by a human operator. No such capability of a human operator is necessary, or desirable in most cases, in any of the operations described herein.

In various embodiments, the embodiments are directed toward one or more computer systems capable of carrying out the functionalities described herein. The computer system includes one or more processors. The processor is connected to a communication infrastructure (e.g., a communications bus, cross-over bar, network, etc.). Various software embodiments are described in terms of this exemplary computer system. After reading this description, it will become apparent to a person skilled in the relevant art(s) how to implement various embodiments using other computer systems and/or architectures. The computer system can include a display interface that forwards graphics, text, and other data from the communication infrastructure (or from a frame buffer not shown) for display on a display unit.

The computer system also includes a main memory, such as random access memory (RAM), and may also include a secondary memory. The secondary memory may include, for example, a hard disk drive, a solid-state drive, and/or a removable storage drive. The removable storage drive reads from and/or writes to a removable storage unit in a well-known manner. As will be appreciated, the removable storage unit includes a computer usable storage medium having stored therein computer software and/or data.

In various embodiments, secondary memory may include other similar devices for allowing computer programs or other instructions to be loaded into a computer system. Such devices may include, for example, a removable storage unit and an interface. Examples of such may include a program cartridge and cartridge interface (such as that found in video game devices), a removable memory chip (such as an erasable programmable read only memory (EPROM), programmable read only memory (PROM)) and associated socket, or other removable storage units and interfaces, which allow software and data to be transferred from the removable storage unit to a computer system.

The terms “computer program medium,” “computer usable medium,” and “computer readable medium” are used to generally refer to media such as removable storage drive and a hard disk installed in hard disk drive. These computer program products provide software to a computer system.

The computer system may also include a communications interface. A communications interface allows software and data to be transferred between the computer system and external devices. Examples of communications interface may include a modem, a network interface (such as an Ethernet card), a communications port, etc. Software and data transferred via the communications interface are in the form of signals which may be electronic, electromagnetic, optical, or other signals capable of being received by communications interface. These signals are provided to communications interface via a communications path (e.g., channel). This channel carries signals and may be implemented using wire, cable, fiber optics, a telephone line, a cellular link, a radio frequency (RF) link, wireless and other communications channels.

Encryption may be performed by way of any of the techniques now available in the art or which may become available—e.g., Twofish, RSA, El Gamal, Schorr signature, DSA, PGP, PM, GPG (GnuPG), HPE Format-Preserving Encryption (FPE), Voltage, Triple DES, Blowfish, AES, MDS, HMAC, IDEA, RC6, and symmetric and asymmetric cryptosystems. The systems and methods may also incorporate SHA series cryptographic methods, elliptic curve cryptography (e.g., ECC, ECDH, ECDSA, etc.), and/or other post-quantum cryptography algorithms under development.

Any databases discussed herein may include relational, hierarchical, graphical, blockchain, object-oriented structure, and/or any other database configurations. Any database may also include a flat file structure wherein data may be stored in a single file in the form of rows and columns, with no structure for indexing and no structural relationships between records. For example, a flat file structure may include a delimited text file, a CSV (comma-separated values) file, and/or any other suitable flat file structure. Common database products that may be used to implement the databases include DB2® by IBM® (Armonk, N.Y.), various database products available from ORACLE® Corporation (Redwood Shores, Calif.), MICROSOFT ACCESS® or MICROSOFT SQL SERVER® by MICROSOFT® Corporation (Redmond, Wash.), MYSQL® by MySQL AB (Uppsala, Sweden), MONGODB®, Redis, APACHE CASSANDRA®, HBASE® by APACHE®, MapR-DB by the MAPR® corporation, or any other suitable database product. Moreover, any database may be organized in any suitable manner, for example, as data tables or lookup tables. Each record may be a single file, a series of files, a linked series of data fields, or any other data structure.

Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors. Various database tuning steps are contemplated to optimize database performance. For example, frequently used files such as indexes may be placed on separate file systems to reduce In/Out (“I/O”) bottlenecks.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked by using AGREP, for example. In accordance with one embodiment, any suitable data storage technique may be utilized to store data without a standard format. Data sets may be stored using any suitable technique, including, for example, storing individual files using an ISO/IEC 7816-4 file structure; implementing a domain whereby a dedicated file is selected that exposes one or more elementary files containing one or more data sets; using data sets stored in individual files using a hierarchical filing system; data sets stored as records in a single file (including compression, SQL accessible, hashed via one or more keys, numeric, alphabetical by first tuple, etc.); data stored as Binary Large Object (BLOB); data stored as ungrouped data elements encoded using ISO/IEC 7816-6 data elements; data stored as ungrouped data elements encoded using ISO/IEC Abstract Syntax Notation (ASN.1) as in ISO/IEC 8824 and 8825; other proprietary techniques that may include fractal compression methods, image compression methods, etc.

In various embodiments, the ability to store a wide variety of information in different formats is facilitated by storing the information as a BLOB. Thus, any binary information can be stored in a storage space associated with a data set. As discussed above, the binary information may be stored in association with the system or external to but affiliated with system. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data, in the database or associated with the system, by multiple and unrelated owners of the data sets. For example, a first data set which may be stored may be provided by a first party, a second data set which may be stored may be provided by an unrelated second party, and yet a third data set which may be stored, may be provided by an third party unrelated to the first and second party. Each of these three exemplary data sets may contain different information that is stored using different data storage formats and/or techniques. Further, each data set may contain subsets of data that also may be distinct from other subsets.

As stated above, in various embodiments, the data can be stored without regard to a common format. However, the data set (e.g., BLOB) may be annotated in a standard manner when provided for manipulating the data in the database or system. The annotation may comprise a short header, trailer, or other appropriate indicator related to each data set that is configured to convey information useful in managing the various data sets. For example, the annotation may be called a “condition header,” “header,” “trailer,” or “status,” herein, and may comprise an indication of the status of the data set or may include an identifier correlated to a specific issuer or owner of the data. In one example, the first three bytes of each data set BLOB may be configured or configurable to indicate the status of that particular data set; e.g., LOADED, INITIALIZED, READY, BLOCKED, REMOVABLE, or DELETED. Subsequent bytes of data may be used to indicate for example, the identity of the issuer, user, transaction/membership account identifier or the like. Each of these condition annotations are further discussed herein.

One skilled in the art will also appreciate that, for security reasons, any databases, systems, devices, servers, or other components of the system may consist of any combination thereof at a single location or at multiple locations, wherein each database or system includes any of various suitable security features, such as firewalls, access codes, encryption, decryption, compression, decompression, and/or the like.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The computer based system 216 may comprise a distributed computing cluster which may be, for example, a HADOOP® software cluster configured to process and store big data sets with some of nodes comprising a distributed storage system and some of nodes comprising a distributed processing system. In that regard, distributed computing cluster may be configured to support a HADOOP® software distributed file system (HDFS) as specified by the Apache Software Foundation at www.hadoop.apache.org/docs.

“Cloud” or “Cloud computing” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand. For more information regarding cloud computing, see the NIST's (National Institute of Standards and Technology) definition of cloud computing at www.csrc.nist.gov/publications/nistpubs/800-145/SP800-145 (last visited June 2012), which is hereby incorporated by reference in its entirety.

As used herein, “transmit” may include sending electronic data from one system component to another over a network connection. Additionally, as used herein, “data” may include encompassing information such as commands, queries, files, data for storage, and the like in digital or any other form.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In re Nuijten to fall outside the scope of patentable subject matter under 35 U.S.C. § 101.

Benefits, other advantages, and solutions to problems have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the inventions. The scope of the inventions is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

Systems, methods and apparatus are provided herein. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A method comprising:
receiving, by a processor, first image data comprising first bioluminescence data derived from an organism at a first time;
receiving, by the processor, second image data comprising second bioluminescence data derived from an organism at a second time;
comparing, by the processor, the first image data to the second image data;
determining, by the processor, whether a peak light output event has occurred based on the comparison; and
outputting, by the processor and to a display device, an indication that the peak light output event has occurred.

2. The method of claim 1, further comprising commanding, by the processor, an optical imaging device to cease imaging activity.

3. The method of claim 1, further comprising defining, by the processor, a region of interest;
identifying, by the processor, the region of interest in the first image data;
determining, by the processor, a first light output associated with the region of interest in the first image data;
identifying, by the processor, the region of interest in the second image data;
determining, by the processor, a second light output associated with the region of interest in the second image data;
comparing, by the processor, the first light output to the second light output.

4. The method of claim 3, further comprising defining, by the processor, a second region of interest;
identifying, by the processor, the second region of interest in the first image data;
determining, by the processor, a third light output associated with the second region of interest in the first image data;
identifying, by the processor, the second region of interest in the second image data;
determining, by the processor, a fourth light output associated with the second region of interest in the second image data;
comparing, by the processor, the third light output to the fourth light output.

5. The method of claim 4, further comprising determining, by the processor, whether a second peak light output event has occurred in the region of interest; and
determining, by the processor, whether a third peak light output event has occurred in the second region of interest.

6. The method of claim 4, wherein the determining, by the processor, whether the peak light output event has occurred comprises subtracting, the processor, a first light output intensity of the region of interest from the second image data from the a second light output intensity of the region of interest from the first image data.

7. The method of claim 5, further comprising commanding, by the processor, an optical imaging device to capture third image data.

8. An article of manufacture including a non-transitory, tangible computer readable storage medium having instructions stored thereon that, in response to execution by a computer-based system, cause the computer-based system to perform operations comprising:
receiving, by the computer-based system, first image data comprising first bioluminescence data derived from an organism at a first time;
receiving, by the computer-based system, second image data comprising second bioluminescence data derived from an organism at a second time;
comparing, by the computer-based system, the first image data to the second image data;
determining, by the computer-based system, whether a peak light output event has occurred based on the comparison; and
outputting, by the computer-based system and to a display device, an indication that the peak light output event has occurred.

9. The article of manufacture of claim 8, further comprising commanding, by the computer-based system, an optical imaging device to cease imaging activity.

10. The article of manufacture of claim 8, further comprising defining, by the computer-based system, a region of interest;
identifying, by the computer-based system, the region of interest in the first image data;
determining, by the computer-based system, a first light output associated with the region of interest in the first image data;
identifying, by the computer-based system, the region of interest in the second image data;
determining, by the computer-based system, a second light output associated with the region of interest in the second image data;
comparing, by the computer-based system, the first light output to the second light output.

11. The article of manufacture of claim 10, further comprising defining, by the computer-based system, a second region of interest;
identifying, by the computer-based system, the second region of interest in the first image data;
determining, by the computer-based system, a third light output associated with the second region of interest in the first image data;
identifying, by the computer-based system, the second region of interest in the second image data;
determining, by the computer-based system, a fourth light output associated with the second region of interest in the second image data;
comparing, by the computer-based system, the third light output to the fourth light output.

12. The article of manufacture of claim 11, further comprising determining, by the computer-based system, whether a second peak light output event has occurred in the region of interest; and
determining, by the computer-based system, whether a third peak light output event has occurred in the second region of interest.

13. The article of manufacture of claim 11, wherein the determining, by the computer-based system, whether the peak light output event has occurred comprises subtracting, the computer-based system, a first light output intensity of the region of interest from the second image data from the a second light output intensity of the region of interest from the first image data.

14. The article of manufacture of claim 12, further comprising commanding, by the computer-based system, an optical imaging device to capture third image data.

15. A system comprising:
a processor;
an optical imaging device;
a display device, the processor in logical communication with the optical imaging device and the display device; and
a tangible, non-transitory memory configured to communicate with the processor,
the tangible, non-transitory memory having instructions stored thereon that, in response to execution by the processor, cause the processor to perform operations comprising:
receiving, by the processor, first image data comprising first bioluminescence data derived from an organism at a first time;
receiving, by the processor, second image data comprising second bioluminescence data derived from an organism at a second time;
comparing, by the processor, the first image data to the second image data;
determining, by the processor, whether a peak light output event has occurred based on the comparison; and
outputting, by the processor and to the display device, an indication that the peak light output event has occurred.

16. The system of claim 15, further comprising commanding, by the processor, the optical imaging device to cease imaging activity.

17. The system of claim 15, further comprising defining, by the processor, a region of interest;
identifying, by the processor, the region of interest in the first image data;
determining, by the processor, a first light output associated with the region of interest in the first image data;
identifying, by the processor, the region of interest in the second image data;
determining, by the processor, a second light output associated with the region of interest in the second image data;
comparing, by the processor, the first light output to the second light output.

18. The system of claim 17, further comprising defining, by the processor, a second region of interest;
identifying, by the processor, the second region of interest in the first image data;
determining, by the processor, a third light output associated with the second region of interest in the first image data;
identifying, by the processor, the second region of interest in the second image data;
determining, by the processor, a fourth light output associated with the second region of interest in the second image data;
comparing, by the processor, the third light output to the fourth light output.

19. The system of claim 18, further comprising determining, by the processor, whether a second peak light output event has occurred in the region of interest; and
determining, by the processor, whether a third peak light output event has occurred in the second region of interest.

20. The system of claim 18, wherein the determining, by the processor, whether the peak light output event has occurred comprises subtracting, the processor, a first light output intensity of the region of interest from the second image data from the a second light output intensity of the region of interest from the first image data.
